(19) United States
(12) Patent Application Publication
(10) Pub. No.: US 2010/0303869 A1
(43) Pub. Date: Dec. 2, 2010

(54) Process for Producing Antimicrobial-Coated Superabsorbents

(75) Inventors: Michael M. Azad, Charlotte, NC (US); James C. Robinson, Weddington, NC (US); Michael Young, Charlotte, NC (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: BASF SE, Ludwigshafen (DE)

(21) Appl. No.: 12/675,578

(22) PCT Filed: Sep. 12, 2008
(86) PCT No.: PCT/EP2008/062111

§ 371 Date: Feb. 26, 2010

Related U.S. Application Data
(60) Provisional application No. 60/971,953, filed on Sep. 13, 2007.

(51) Int. Cl. A61K 9/00; A01N 31/14; A01P 1/00

(52) U.S. Cl. 424/400; 514/721

(57) ABSTRACT

Superabsorbents having a coating of an antimicrobial agent are produced by a process that comprises contacting the superabsorbent with a solution comprising the anti-microbial agent and a polyol concurrently with or immediately after contacting it with the surface-crosslinking agent and prior to the curing step that completes surface crosslinking, and/or contacting the superabsorbent with a solution comprising the anti-microbial agent and polyalkylene glycol of a molecular mass between 200 and 5 000 g/mol after completion of surface crosslinking.

The present invention relates to a process for producing superabsorbents that are coated with an antimicrobial agent.

Superabsorbents are known. Superabsorbents are materials that are able to take up and retain several times their weight in water, possibly up to several hundred times their weight, even under moderate pressure. Absorbing capacity is usually lower for salt-containing solutions compared to distilled or otherwise de-ionised water. Typically, a superabsorbent has a centrifugal retention capacity (“CRC”, method of measurement see hereinbelow) of at least 5 g/g, preferably at least 10 g/g and more preferably at least 15 g/g. Such materials are also commonly known by designations such as “high-swellability polymer”, “hydrogel” (often even used for the dry form), “hydrogel-forming polymer”, “water-absorbing polymer”, “absorbent gel-forming material”, “swellable resin”, “water-absorbing resin” or the like. The materials in question are crosslinked hydrophilic polymers, in particular polymers formed from (co)polymerized hydrophilic monomers, graft (co)polymers of one or more hydrophilic monomers on a suitable grafting base, crosslinked ethers of cellulose or starch, crosslinked carboxymethylcellulose, partially crosslinked polyalkylene oxide or natural products that are swellable in aqueous fluids, examples being guar derivatives, of which water-absorbing polymers based on partially neutralized acrylic acid are most widely used. Superabsorbents are usually produced, stored, transported and processed in the form of dry powders of polymer particles, “dry” usually meaning less than 5 wt.-% water content, although forms in which superabsorbents particles are bound to a web, typically a nonwoven, are also known for some applications, as are superabsorbent fibres. A superabsorbent transforms into a gel on taking up a liquid, specifically into a hydrogel when as usual taking up water. By far the most important field of use of superabsorbents is the absorbing of bodily fluids. Superabsorbents are used for example in diapers for infants, incontinence products for adults or feminine hygiene products. Examples of other fields of use are as water-retaining agents in market gardening, as water stores for protection against fire, for liquid absorption in food packaging or, in general, for absorbing moisture.

Processes for producing superabsorbents are also known. The acrylate-based superabsorbents which dominate the market are produced by radical polymerization of acrylic acid in the presence of a crosslinking agent (the “internal crosslinker”), usually in the presence of water, the acrylic acid being neutralized to some degree in a neutralization step conducted prior to or after polymerization, or optionally partly prior to and partly after polymerization, usually by adding a alkali, most often an aqueous sodium hydroxide solution. This yields a polymer gel which is comminuted (depending on the type of reactor used, comminution may be conducted concurrently with polymerization) and dried. Usually, the dried powder thus produced (the “base polymer”) is surface crosslinked (also termed surface “post”crosslinked) by adding further organic or polyvalent cationic crosslinkers to generate a surface layer which is crosslinked to a higher degree than the particle bulk. Most often, aluminium sulphate is being used as polyvalent cationic crosslinker. Applying polyvalent metal cations to superabsorbent particles is sometimes not regarded as surface crosslinking, but termed “surface complexing” or as another form of surface treatment, although it has the same effect of increasing the number of bonds between individual polymer strands at the particle surface and thus increases gel particle stiffness as organic surface crosslinkers have. Organic and polyvalent cation surface crosslinkers can be cumulatively applied, jointly or in any sequence.

Surface crosslinking leads to a higher crosslinking density close to the surface of each superabsorbent particle. This addresses the problem of “gel blocking”, which means that, with earlier types of superabsorbents, a liquid insult will cause swelling of the outermost layer of particles of a bulk of superabsorbent particles into a practically continuous gel layer, which effectively blocks transport of further amounts of liquid (such as a second insult) to unused superabsorbent below the gel layer. While this is a desired effect in some applications of superabsorbents (for example sealing underwater cables), it leads to undesirable effects when occurring in personal hygiene products. Increasing the stiffness of individual gel particles by surface crosslinking leads to open channels between the individual gel particles within the gel layer and thus facilitates liquids transport through the gel layer. Although surface crosslinking decreases the CRC or other parameters describing the total absorption capacity of a superabsorbent sample, it may well increase the amount of liquid that can be absorbed by hygiene product containing a given amount of superabsorbent.

Other means of increasing the permeability of a superabsorbent are also known. These include admixing of superabsorbent with fibres such as fluff in a diaper core or admixing other components that increase gel stiffness or otherwise create open channels for liquid transportation in a gel layer.

Frederic L. Buchholz and Andrew T. Graham (Eds.) in: “Modern Superabsorbent Polymer Technology”, J. Wiley & Sons, New York, U.S.A./Wiley-VCH, Weinheim, Germany, 1997, ISBN 0-471-19411-5, give a comprehensive overview over superabsorbents and processes for producing superabsorbents.

When superabsorbents are used in the hygiene sector, they are exposed to bodily fluids such as urine or menses. Such bodily fluids always contain malodorous components such as amines, fatty acids and other organic components which are responsible for unpleasant body odours. A further problem with such hygiene products is that the bodily fluids remain in the hygiene product for a certain time until the hygiene product is disposed of, and bacterial degradation of nitrogenous compounds present in the absorbed bodily fluids, an example being urea in urine, gives rise to ammonia or else other amines which likewise lead to a noticeable odour nuisance. Since correspondingly frequent changing of the hygiene product leads to an appreciable inconvenience and also cost for the user or his or her care persons, hygiene products where this odour nuisance is avoided are of advantage.

Various measures to avoid the odour nuisance are known. Odours can be masked by perfumes; the ammonia which results or amines can be removed by absorption or reaction, and the microbial degradation can be inhibited by means of biocides or urease inhibitors for example. These measures can be applied to the superabsorbent on the one hand and to the hygiene article on the other.

For instance, EP 1 358 894 A1 teaches hygiene articles which may include a series of odour-preventing additives, in particular anhydride groups, acid groups, cyclodextrins, biocides, surfactants having an HLB value of less than 11, absorbents such as zeolithes, clay, activated carbon, silica or activated alumina, micro organisms which act as antagonists to undesirable odour-forming micro organisms, pH buffers or chelating agents. WO 03/076 514 A2 features a comprehensive overview of existing measures for avoiding unpleasant odours. The use of biocides such as bronopol or glyoxylic acid is disclosed among other measures. In addition, this reference teaches a superabsorbent containing anhydride groups capable of reacting with ammonia or amines and binding them in non-volatile form as a result.

EP 739 635 A1 teaches sodium metaborate and sodium tetraborate useful as urease inhibitors in superabsorbents. WO 94/25 077 A1 utilizes a mixture of alkali metal or alkaline earth metal tetraborate and boric acid, citric acid, tartaric acid or ascorbic acid as a buffer in pH range from 7 to 10. WO 03/053486 A1 discloses diapers comprising yucca palm extract as urease inhibitor. EP 1 214 878 A1 discloses the use of chelate complexes of bivalent metal ions such as of the copper complex of singly proteinated ethylenediaminetetraacetate as a urease inhibitor. WO 95/24173 A2 teaches the use of zeolithes impregnated with bactericidal heavy metals such as silver, zinc or copper for odour control.

EP 311 344 A23 concerns hygiene articles which, as well as a pH buffer, comprise a biocide such as alkyl ammonium halides or bisguanidines. EP 389 023 A2 discloses hygiene articles comprising an odour control additive selected from biocides or absorbents, in particular molecular sieves. WO 98/26 808 A2 describes superabsorbents comprising cyclodextrins, zeolithes, activated carbon, diatomaceous earth or acidic salt-forming substances as absorbents for odours and also biocides, urease inhibitors and pH regulators to inhibit odour formation.

WO 2007/012 581 A1 concerns storage-stable superabsorbents comprising substituted thiophosphoramides as odour inhibitor. The thiophosphoramides are coated on the superabsorbent particles by spraying a solution of the thiophosphoramide in water, water-acetone or water-propylene glycol solvent on the superabsorbent at any time during its production, for example during the surface-crosslinking step.

WO 98/20 916 A1 utilizes a superabsorbent in hygiene articles which is coated with an antimicrobial biocide, among the listed examples of preferred antimicrobials are common biocides such as benzalkonium chloride, triclosan (common name for 2,4,4′-trichloro-2′-hydroxidiphenyl ether), sodium parabens, 2,4-imidazolidinedione, citric and sorbic acid or bronopol (common name for 2-bromo-2-nitropropane-1,3-diol). The antimicrobial is applied to the superabsorbent in solution, preferably in aqueous solution, and if insoluble in water, using a polar organic solvent such as methanol, ethanol or propanol, acetone, dimethylformamide, dimethylsulfoxide, hexamethylphosphoric triamide or mixtures thereof, or mixtures thereof with water, or using a non-polar solvent such as hydrocarbons, by contacting the superabsorbent with this solution to form a coating, followed by solvent removal to produce a antimicrobial-coated superabsorbent. The anti-microbial is applied to the superabsorbent before, concurrently with or after surface crosslinking.

WO 00/66 187 A1 discloses a superabsorbent polymer comprising an odour-controlling compound distributed homogeneously throughout the superabsorbent particles. The odour-controlling compound is a cyclodextrin, an amphoteric surfactant, a water-insoluble phosphate, triclosan or a mixture thereof. This superabsorbent is produced either by polymerising a monomer solution that also comprises the odour-controlling compound or, particularly if the odour-controlling compound is unstable under polymerisation conditions, such as triclosan, by mixing the wet gel obtained from solution polymerization of the monomers with the compound prior to drying and any further treatment.

WO 01/44 355 A1 teaches a superabsorbent that contains an anti-microbial additive, in particular 0.01 to 0.5 wt.-% triclosan, having improved stability against sedimentation or separation of triclosane from the superabsorbent, and a process for its production. In this process, triclosan is either mixed into to the superabsorbent and the mixture then heated to a temperature of at least 5° C. above the melting point of triclosan or by first heating a mixture of superabsorbent and triclosan to make a masterbatch containing about 5 wt.-% triclosan and then mixing this masterbatch with superabsorbent to make the final product.

WO 2004/020 514 A2 discloses acrylic polymers, in particular termoformable polymethycrylic acid polymer sheets, comprising triclosan and orthophenyl phenol to impart anti-microbial properties.

Despite the advanced state of the art as outlined in the cited prior art, there still is a need for improved processes for producing superabsorbents coated with an anti-microbial agent. Such processes are desired to be simple and yield a superabsorbent with an at least reasonably uniform coating of a sufficient amount of anti-microbial agent in a simple and economic way without interference with the superabsorbents production itself or the need for additional process steps or equipment. It is an object of this invention to find such a process.

This object has been solved by a process for producing superabsorbents having a coating of an antimicrobial agent that comprises contacting the superabsorbent with a solution comprising the anti-microbial agent and a polyol concurrently with or immediately after contacting it with the surface-crosslinking agent and prior to the curing step that completes surface crosslinking, and/or contacting the superabsorbent with a solution comprising the anti-microbial agent and polyalkylene glycol of a molecular mass between 200 and 5 000 g/mol after completion of surface crosslinking.

The anti-microbial agent is any microbial agent known in the art to be effective for inhibiting growth of odour-generating micro organisms in applications of superabsorbents. Examples are quaternary ammonium compounds such as benzalkonium chloride or poly(hexamethylenebiguanide) hydrochloride (“PHMB”, an oligomeric compound of the formula —[CH₂—CH₂—CH₂—NH—CNH—NH—CNH₂Cl—NH—CH₂—CH₂—CH₂]_{n—, n being from} 10 to 20, and in commercial products on average 16, available under the trademarks Reputex® 20or Purista® 20from Arch Chemicals Inc., Norwalk , Conn., USA, or BASF Aktiengesellschaft, Ludwigshafen, Germany), phenol derivatives such as triclosan, phenoxiethanol or parabens, acids such as benzoic acid, citric acid and sorbic acid, nitro compounds such as bronopol, metal ion compounds, in particular silver compounds or compounds that release metal ions, in particular silver ions, isothiazolones such as 2-methyl-3-(2H)-isothiazolone or 5-chloro-2-methyl-3-(2H)-isothiazolone, or mixtures thereof. A particularly preferred anti-microbial agent is triclosan.

The anti-microbial agent is generally added in an amount of at least 100 wt.-ppm, preferably at least 200 wt.-ppm, more preferably at least 300 wt.-ppm and generally at most 5000 wt.-ppm, preferably at most 3000 wt.-ppm and more preferably at most 1500 wt.-ppm, in each case based on the total weight of material.

The superabsorbent in the present invention is a superabsorbent capable of absorbing and retaining amounts of water equivalent to many times its own weight under a certain pressure. In general, it has a centrifugal retention capacity (CRC, method of measurement see hereinbelow) of at least 5 g/g, preferably at least 10 g/g and more preferably at least 15 g/g. Preferably, the superabsorbent is a crosslinked polymer based on partially neutralized acrylic acid and more preferably it is surface postcrosslinked. A “superabsorbent” can also be a mixture of chemically different individual superabsorbents in that it is not so much the chemical composition which matters as the superabsorbing properties.

Processes for producing superabsorbents, including surface-postcrosslinked superabsorbents, are known. Synthetic superabsorbents are obtained for example by polymerization of a monomer solution comprising
a) at least one ethylenically unsaturated acid-functional monomer,
b) at least one crosslinker,
c) optionally one or more ethylenically and/or allylically unsaturated monomers co-polymerizable with the monomer a), and
d) optionally one or more water-soluble polymers onto which the monomers a), b) and if appropriate c) can be at least partly grafted.

Suitable monomers a) are for example ethylenically unsaturated carboxylic acids, such as acrylic acid, methacrylic acid, maleic acid, fumaric acid and itaconic acid, or derivatives thereof, such as acrylamide, methacrylamide, acrylic esters and methacrylic esters. Acrylic acid and methacrylic acid are particularly preferred monomers. Acrylic acid is most preferable.

The monomers a) and especially acrylic acid comprise preferably up to 0.025% by weight of a hydroquinone half ether. A preferred hydroquinone half ether is hydroquinone monomethyl ether (MEHQ). Tocopherols such as alpha-tocopherol, especially racemic alpha-tocopherol or RRR-alpha-tocopherol are also preferred.

The monomer solution comprises preferably not more than 130 weight ppm, more preferably not more than 70 weight ppm, preferably not less than 10 weight ppm, more preferably not less than 30 weight ppm and especially about 50 weight ppm of hydroquinone half ether, all based on acrylic acid, with acrylic acid salts being arithmetically counted as acrylic acid. For example, the monomer solution can be produced using an acrylic acid having an appropriate hydroquinone half ether content.

Crosslinkers b) are compounds having at least two polymerizable groups which can be free-radically interpolymerized into the polymer network. Useful crosslinkers b) include for example ethylene glycol dimethacrylate, diethylene glycol diacrylate, allyl methacrylate, trimethylolpropane triacrylate, triallylamine, tetraallyloxyethane as described in EP 530 438 A1, di- and triacrylates as described in EP 547 847 A1, EP 559 476 A1, EP 632 068 A1, WO 93/21 237 A1, WO 03/104 299 A1, WO 03/104 300 A1, WO 03/104 301 A1 and DE 103 31 450 A1, mixed acrylates which, as well as acrylate groups, comprise further ethylenically unsaturated groups, as described in DE 103 31 456 A1 and WO 04/013 064 A2, or crosslinker mixtures as described for example in DE 195 43 368 A1, DE 196 46 484 A1, WO 90/15 830 A1 and WO 02/032 962 A2.

Useful crosslinkers b) include in particular N,N′-methylenebisacrylamide and N,N′-methylenebismethacrylamide, esters of unsaturated mono- or polycarboxylic acids of polyols, such as diacrylate or triacrylate, for example butanediol diacrylate, butanediol dimethacrylate, ethylene glycol diacrylate, ethylene glycol dimethacrylate and also trimethylolpropane triacrylate and allyl compounds, such as allyl (meth)acrylate, triallyl cyanurate, diallyl maleate, polyallyl esters, tetraallyloxyethane, triallylamine, tetraallylethylenediamine, allyl esters of phosphoric acid and also vinylphosphonic acid derivatives as described for example in EP 343 427 A2. Useful crosslinkers b) further include pentaerythritol diallyl ether, pentaerythritol triallyl ether, pentaerythritol tetraallyl ether, polyethylene glycol diallyl ether, ethylene glycol diallyl ether, glycerol diallyl ether, glycerol triallyl ether, polyallyl ethers based on sorbitol, and also ethoxylated variants thereof. The process of the present invention may utilize di(meth)acrylates of polyethylene glycols, the polyethylene glycol used having a molecular weight between 300 and 1000.

However, particularly advantageous crosslinkers b) are di- and triacrylates of 3- to 15-tuply ethoxylated glycerol, of 3- to 15-tuply ethoxylated trimethylolpropane, of 3- to 15-tuply ethoxylated trimethylolethane, especially di- and triacrylates of 2- to 6-tuply ethoxylated glycerol or of 2- to 6-tuply ethoxylated trimethylolpropane, of 3-tuply propoxylated glycerol, of 3-tuply propoxylated trimethylolpropane, and also of 3-tuply mixedly ethoxylated or propoxylated glycerol, of 3-tuply mixedly ethoxylated or propoxylated trimethylolpropane, of 15-tuply ethoxylated glycerol, of 15-tuply ethoxylated trimethylolpropane, of 40-tuply ethoxylated glycerol, of 40-tuply ethoxylated trimethylolethane and also of 40-tuply ethoxylated trimethylolpropane.

Very particularly preferred for use as crosslinkers b) are diacrylated, dimethacrylated, triacrylated or trimethacrylated multiply ethoxylated and/or propoxylated glycerols as described for example in WO 03/104 301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. The triacrylates of 3- to 5-tuply ethoxylated and/or propoxylated glycerol are most preferred. These are notable for particularly low residual contents (typically below 10 weight ppm) in the water-absorbing polymer and the aqueous extracts of the water-absorbing polymers produced therewith have an almost unchanged surface tension (typically at least 0.068 N/m) compared with water at the same temperature.

Examples of ethylenically unsaturated monomers c) which are copolymerizable with the monomers a) are acrylamide, methacrylamide, crotonamide, dimethylaminoethyl methacrylate, dimethylaminoethyl acrylate, dimethylaminopropyl acrylate, diethylaminopropyl a crylate, dimethylaminobutyl acrylate, dimethylaminoethyl methacrylate, diethylaminoethyl methacrylate, dimethylaminoneopentyl acrylate and dimethylaminoneopentyl methacrylate.

Useful water-soluble polymers d) include polyvinyl alcohol, polyvinylpyrrolidone, starch, starch derivatives, polyethyleneimines, polyglycols, polymers formally constructed wholly or partly of vinylamine monomers, such as partially or completely hydrolyzed polyvinylamide (so-called “polyvinylamine”) or polyacrylic acids, preferably polyvinyl alcohol and starch.

The polymerization is optionally carried out in the presence of customary polymerization regulators. Suitable polymerization regulators are for example thio compounds, such as thioglycolic acid, mercapto alcohols, for example 2-mercaptoethanol, mercaptopropanol and mercaptobutanol, dodecyl mercaptan, formic acid, ammonia and amines, for example ethanolamine, diethanolamine, triethanolamine, triethylamine, morpholine and piperidine.

The monomers (a), (b) and optionally (c) are (co)polymerized with each other in the presence of the water-soluble polymers d), in 20% to 80%, preferably 20% to 50% and especially 30% to 45% by weight aqueous solution in the presence of polymerization initiators. Useful polymerization initiators include all compounds that disintegrate into free radicals under the polymerization conditions, examples being peroxides, hydroperoxides, hydrogen peroxide, persulfates, azo compounds and the so-called redox initiators. The use of water-soluble initiators is preferred. It is advantageous in some cases to use mixtures of various polymerization initiators, examples being mixtures of hydrogen peroxide and sodium or potassium peroxodisulfate. Mixtures of hydrogen peroxide and sodium peroxodisulfate can be used in any desired ratio. Suitable organic peroxides are for example acetylacetone peroxide, methyl ethyl ketone peroxide, tert-butyl hydroperoxide, cumene hydroperoxide, tert-amyl perpivalate, tert-butyl perpivalate, tert-butyl perneohexanoate, tert-butyl perisobutyrate, tert-butyl per-2-ethylhexanoate, tert-butyl perisononanoate, tert-butyl permaleate, tert-butyl perbenzoate, tert-butyl per-3,5,5-trimethylhexanoate and tert-amyl perneodecanoate. Further suitable polymerization initiators are azo initiators, for example 2,2′-azobis(2-amidinopropane) dihydrochloride, 2,2′-azobis(N,N-dimethylene)-isobutyramidine dihydrochloride, 2-(carbamoylazo)isobutyronitrile and 4,4′-azobis(4-cyanovaleric acid). The polymerization initiators mentioned are used in customary amounts, for example in amounts of from 0.01 to 5 mol %, preferably 0.1 to 2 mol %, based on the monomers to be polymerized.

The redox initiators comprise, as oxidizing component, at least one of the above-indicated per compounds and a reducing component, for example ascorbic acid, glucose, sorbose, ammonium bisulfite, ammonium sulfite, ammonium thiosulfate, ammonium hyposulfite, ammonium pyrosulfite, ammonium sulfide, alkali metal bisulfite, alkali metal sulfite, alkali metal thiosulfate, alkali metal hyposulfite, alkali metal pyrosulfite, alkali metal sulfide, metal salts, such as iron(II) ions or silver ions, sodium hydroxymethylsulfoxylate, or sulfinic acid derivatives. The reducing component of the redox initiator is preferably ascorbic acid or sodium pyrosulfite. 1.10⁻⁵ to 1 mol % of the reducing component of the redox initiator and 1.10⁻⁵ to 5 mol % of the oxidizing component are used based on the amount of monomers used in the polymerization. Instead of the oxidizing component or in addition it is also possible to use one or more water-soluble azo initiators.

A redox initiator consisting of hydrogen peroxide, sodium peroxodisulfate and ascorbic acid is preferably used. These components are used for example in the concentrations of 1·10⁻² mol % of hydrogen peroxide, 0.084 mol % of sodium peroxodisulfate and 2.5·10⁻³ mol % of ascorbic acid, based on the monomers.

It is also possible to initiate the polymerization by the numerous other known means to initiate polymerizations. On example is initiating polymerization by irradiating with radiation of sufficiently high energy, in particular ultraviolet light. Usually, when initiating polymerization by ultraviolet light, compounds are added which decompose into radicals upon irradiation by ultraviolet light. Examples of such compunds are 2-hydroxi-2-methyl-1-phenyl-1-propanone and/or alpha,-alpha-dimethoxi-alpha-phenylacetophenone.

The aqueous monomer solution may comprise the initiator in dissolved or dispersed form. However, the initiators may also be added to the polymerization reactor separately from the monomer solution.

The preferred polymerization inhibitors require dissolved oxygen for optimum effect. Therefore, the polymerization inhibitors can be freed of dissolved oxygen prior to polymerization, by inertization, i.e., by flowing an inert gas, preferably nitrogen, through them. This is accomplished by means of inert gas, which can be introduced concurrently, countercurrently or at entry angles in between. Good commixing can be achieved for example with nozzles, static or dynamic mixers or bubble columns. The oxygen content of the monomer solution is preferably lowered to less than 1 weight ppm and more preferably to less than 0.5 weight ppm prior to polymerization. The monomer solution is optionally passed through the reactor using an inert gas stream.

The preparation of a suitable polymer as well as further suitable hydrophilic ethylenically unsaturated monomers a) are described for example in DE 199 41 423 A1, EP 686 650 A1, WO 01/45 758 A1 and WO 03/104 300 A1.

Superabsorbents are typically obtained by addition polymerization of an aqueous monomer solution and optionally a subsequent comminution of the hydrogel. Suitable methods of making are described in the literature. Superabsorbents are obtained for example by
gel polymerization in the batch process or tubular reactor and subsequent comminution in meat grinder, extruder or kneader, as described for example in EP 445 619 A2 and DE 19 846 413 A1;
polymerization in kneader with continuous comminution by contrarotatory stirring shafts for example, as described for example in WO 01/38 402 A1;
polymerization on belt and subsequent comminution in meat grinder, extruder or kneader, as described for example in EP 955 086 A2, DE 38 25 366 A1 or U. S. Pat. No. 6,241,928;
emulsion polymerization, which produces bead polymers having a relatively narrow gel size distribution, as described for example in EP 457 660 A1;
in situ polymerization on a woven fabric layer which, usually in a continuous operation, has previously been sprayed with aqueous monomer solution and subsequently been subjected to a photopolymerization, as described for example in WO 02/94 328 A2, WO 02/94 329 A1.

The cited references are expressly incorporated herein for details of process operation. The reaction is preferably carried out in a kneader or on a belt reactor.

Continuous gel polymerization is the economically preferred and therefore currently customary way of manufacturing superabsorbents. The process of continuous gel polymerization is carried out by first producing a monomer mixture by admixing the acrylic acid solution with the neutralizing agent, optional comonomers and/or further auxiliary materials at different times and/or locations and then transferring the mixture into the reactor or preparing the mixture as an initial charge in the reactor. The initiator system is added last to start the polymerization. The ensuing continuous process of polymerization involves a reaction to form a polymeric gel, i.e., a polymer swollen in the polymerization solvent—typically water—to form a gel, and the polymeric gel is already comminuted in the course of a stirred polymerization. The polymeric gel is subsequently dried, if necessary, and also chipped ground and sieved and is transferred for further surface treatment.

The acid groups of the hydrogels obtained are partially neutralized in an acid neutralization step, generally to an extent of at least 25 mol %, preferably to an extent of at least 50 mol % and more preferably at least 60 mol % and generally to an extent of not more than 85 mol %, preferably not more than 80 mol %, and more preferably not more than 75 mol %.

Neutralization can also be carried out after polymerization, at the hydrogel stage. But it is also possible to carry out the neutralization to the desired degree of neutralization wholly or partly prior to polymerization. In the case of partial neutralization and prior to polymerization, generally at least 10 mol %, preferably at least 15 mol % and also generally not more than 40 mol %, preferably not more than 30 mol % and more preferably not more than 25 mol % of the acid groups in the monomers used are neutralized prior to polymerization by adding a portion of the neutralizing agent to the monomer solution.

The desired final degree of neutralization is in this case only set toward the end or after the polymerization, preferably at the level of the hydrogel prior to its drying. The monomer solution is neutralized by admixing the neutralizing agent. The hydrogel can be mechanically comminuted in the course of the neutralization, for example by means of a meat grinder or comparable apparatus for comminuting gellike masses, in which case the neutralizing agent can be sprayed, sprinkled or poured on and then carefully mixed in. To this end, the gel mass obtained can be repeatedly meat-grindered for homogenization.

Neutralization of the monomer solution to the desired final degree of neutralization prior to polymerization by addition of the neutralizing agent or conducting the neutralization after polymerization is usually simpler than neutralization partly prior to and partly after polymerization and therefore is preferred.

The as-polymerized gels are optionally maintained for some time, for example for at least 30 minutes, preferably at least 60 minutes and more preferably at least 90 minutes and also generally not more than 12 hours, preferably for not more than 8 hours and more preferably for not more than 6 hours at a temperature of generally at least 50° C. and preferably at least 70° C. and also generally not more than 130° C. and preferably not more than 100° C., which further improves their properties in many cases.

The neutralized hydrogel is then dried with a belt or drum dryer until the residual moisture content is preferably below 15% by weight and especially below 10% by weight, (the moisture content being determined as described below). The dry superabsorbent consequently contains up to 15% by weight of moisture and preferably not more than 10% by weight. The decisive criterion for classification as “dry” is in particular a sufficient flowability for handling as a powder, for example for pneumatic conveying, pack filling, sieving or other processing steps involved in solids processing technology. Optionally, however, drying can also be carried out using a fluidized bed dryer or a heated ploughshare mixer. To obtain particularly colourless products, it is advantageous to dry this gel by ensuring rapid removal of the evaporating water. To this end, dryer temperature must be optimized, air feed and removal has to be policed, and at all times sufficient venting has to be ensured. Drying is naturally all the more simple—and the product all the more colourless—when the solids content of the gel is as high as possible. The solvent fraction at addition polymerization is therefore set such that the solid content of the gel prior to drying is therefore generally at least 20% by weight, preferably at least 25% by weight and more preferably at least 30% by weight and also generally not more than 90% by weight, preferably not more than 85% by weight and more preferably not more than 80% by weight. It is particularly advantageous to vent the dryer with nitrogen or some other non-oxidizing inert gas. Optionally, however, simply just the partial pressure of oxygen can be lowered during drying to prevent oxidative yellowing processes. But in general adequate venting and removal of the water vapour will likewise still lead to an acceptable product. A very short drying time is generally advantageous with regard to colour and product quality.

The dried hydrogel (which is no longer a gel (even though often still called that) but a dry polymer having superabsorbing properties, which comes within the term “superabsorbent”) is preferably ground and sieved, useful grinding apparatus typically including roll mills, pin mills, hammer mills, cutting mills or swing mills. The particle size of the sieved, dry hydrogel is preferably below 1000 μm, more preferably below 900 μm and most preferably below 850 μm and preferably above 80 μm, more preferably above 90 μm and most preferably above 100 μm.

Very particular preference is given to a particle size (sieve cut) in the range from 106 to 850 μm. Particle size distribution is determined as described below.

The dry superabsorbing polymers thus produced are typically known as “base polymers” and are then preferably surface postcrosslinked. Surface postcrosslinking can be accomplished in a conventional manner using dried, ground and classified polymeric particles. For surface postcrosslinking, compounds capable of reacting with the functional groups of the base polymer by crosslinking are applied, usually in the form of a solution, to the surface of the base polymer particles. Suitable postcrosslinking agents are for example:
di- or polyepoxides, for example di- or polyglycidyl compounds such as phosphonic acid diglycidyl ether , ethylene glycol diglycidyl ether, bischlorohydrin ethers of polyalkylene glycols,
alkoxysilyl compounds,
polyaziridines, compounds comprising aziridine units and based on polyethers or substituted hydrocarbons, for example bis-N-aziridinomethane,
polyamines or polyamidoamines and also their reaction products with epichlorohydrin,
polyols such as ethylene glycol, 1,2-propanediol, 1,4-butanediol, glycerol, methyl-triglycol, polyethylene glycols having an average molecular weight Mw of 200-10,000, di- and polyglycerol, pentaerythritol, sorbitol, the ethoxylates of these polyols and also their esters with carboxylic acids or carbonic acid such as ethylene carbonate or propylene carbonate,
carbonic acid derivatives such as urea, thiourea, guanidine, dicyandiamide, 2-oxazolidinone and its derivatives, bisoxazoline, polyoxazolines, di- and polyisocy-anates,
di- and poly-N-methylol compounds such as for example methylenebis(N-methylolmethacrylamide) or melamine-formaldehyde resins,
compounds having two or more blocked isocyanate groups such as for example trimethylhexamethylene diisocyanate blocked with 2,2,3,6-tetramethylpiperidin-4-one.

If necessary, acidic catalysts can be added, examples being p-toluenesulfonic acid, phosphoric acid, boric acid or ammonium dihydrogenphosphate.

Particularly suitable postcrosslinking agents are di- or polyglycidyl compounds such as ethylene glycol diglycidyl ether, the reaction products of polyamidoamines with epichlorohydrin, 2-oxazolidinone and N-hydroxyethyl-2-oxazolidinone.

Surface postcrosslinking (often just “postcrosslinking”) is typically carried out by spraying a solution of the surface postcrosslinker (often just “postcrosslinker”) onto the hydrogel or the dry base polymer powder.

The solvent used for the surface postcrosslinker is a customary suitable solvent, examples being water, alcohols, DMF, DMSO and also mixtures thereof. Particular preference is given to water and water-alcohol mixtures, examples being water-methanol, water-1,2-propanediol, water-2-propanol and water-1,3-propanediol.

The spraying with a solution of the postcrosslinker is preferably carried out in mixers having moving mixing implements, such as screw mixers, paddle mixers, disk mixers, ploughshare mixers and shovel mixers. Particular preference is given to vertical mixers and very particular preference to ploughshare mixers and shovel mixers. Useful and known mixers include for example Lödige®, Bepex®, Nauta®, Processall® and Schugi® mixers. Very particular preference is given to high speed mixers, for example of the Schugi-Flexomix® or Turbolizer® type.

The spraying with the crosslinker solution can be optionally followed, and generally is, by a thermal treatment step, essentially to effect the surface-postcrosslinking reaction and complete surface crosslinking. This step is sometimes referred to as “curing” or just as “drying”. It is typically performed in a heated mixer (“dryer”) directly downstream from the apparatus used for adding the surface crosslinker, at a temperature of generally at least 50° C., preferably at least 80° C. and more preferably at least 80° C. and also generally not more than 300° C., preferably not more than 250° C. and more preferably not more than 200° C. The average residence time (i.e., the averaged residence time of the individual particles of superabsorbent) in the dryer of the superabsorbent to be treated is generally at least 1 minute, preferably at least 3 minutes and more preferably at least 5 minutes and also generally not more than 6 hours, preferably 2 hours and more preferably not more than 1 hour. As well as the actual drying taking place, not only any products of scissioning present but also solvent fractions are removed. Thermal drying is carried out in customary dryers such as tray dryers, rotary tube ovens or heatable screws, preferably in contact dryers. Preference is given to the use of dryers in which the product is agitated, i.e., heated mixers, more preferably shovel dryers and most preferably disk dryers. Bepex® dryers and Nara® dryers are suitable dryers for example. Fluidized bed dryers can also be used. But drying can also take place in the mixer itself, by heating the jacket or blowing a preheated gas such as air into it. But it is also possible for example to utilize an azeotropic distillation as a drying process. The crosslinking reaction can take place not only before but also during drying.

Additives may be added in this step. Examples of additives that optionally are added in this surface crosslinking step are permeability enhancing agents such as particulate inorganic or organic solids, cationic polymers and water-soluble polyvalent metal salts, or combinations thereof, in the typical total amounts of at least 0.05 wt.-%, preferably at least 0.1 wt.-%, more preferably at least 0.3 wt.-% and generally at most 5 wt.-%, preferably at most 1.5 wt.-% and more preferably at most 1 wt.-%, in each case based on the total weight of the material, and using the typical methods of addition known in the art.

Quite usually, but not necessarily, water-soluble polyvalent metal salts comprise bi- or more highly valent (“polyvalent”) metal cations capable of reacting with the acid groups of the polymer to form complexes are added. Examples of polyvalent cations are or metal cations such as Mg²⁺, Ca²⁺, Al³⁺, Sc³⁺, Ti⁴⁺, Mn²⁺, Fe^{2+/3+}, Co²⁺, Ni²⁺, Cu²⁺, Zn²⁺, Y³⁺, Zr⁴⁺, La³⁺, Ce⁴⁺, Hf⁴⁺, and Au³⁺. Preferred metal cations are Mg²⁺, Ca²⁺, Al³⁺, Ti⁴⁺, Zr⁴⁺ and La³⁺, and particularly preferred metal cations are Al³⁺, Ti⁴⁺ and Zr⁴⁺. The metal cations can be used not only alone but also in admixture with each other. Of the metal cations mentioned, any metal salt can be used that has sufficient solubility in the solvent to be used. Metal salts with weakly complexing anions such as for example chloride, nitrate and sulphate, hydrogen sulphate, carbonate, hydrogen carbonate, nitrate, phosphate, hydrogen phosphate, dihydrogen phosphate and carboxylate, such as acetate and lactate, are particularly suitable. It is particularly preferred to use aluminum sulfate.

The treatment of the superabsorbent polymer with solution of a polyvalent cation is carried out in the same way as that with surface postcrosslinker, including the selective drying step. Useful solvents for the metal salts include water, alcohols, DMF, DMSO and also mixtures thereof. Particular preference is given to water and water-alcohol mixtures such as for example water-methanol, water-1,2-propanediol, water-2-propanol and water-1,3-propanediol.

In a preferred embodiment of the present invention, the permeability enhancing agent is applied to a superabsorbent that is surface crosslinked, or concurrently with surface crosslinking, or partly simultaneously and partly after surface crosslinking. For example, a suitable method of applying a permeability enhancing agent is applying a polyvalent metal cation such as Al³⁺ concurrently with a surface crosslinker and applying a particulate solid such as silica after the step of surface crosslinking, for example during a cooling step conducted after drying the surface crosslinked product.

A first method to add the anti-microbial agent to the superabsorbent is contacting the superabsorbent with a solution comprising the anti-microbial agent and a polyol concurrently with or immediately after contacting it with the surface-crosslinking agent and prior to the curing step that completes surface crosslinking. To this end, a solution of the anti-microbial agent in a solvent that comprises a polyol is added to the superabsorbent. The solution can be added in any known way to achieve a coating of a solution on particles. Any method known or described above to achieve this with respect to a solution of the surface crosslinker is feasible for the solution of the anti-microbial agent as well. Spraying the solution onto the superabsorbent is preferred. Preferably, the solution of the anti-microbial agent is added in the same type of apparatus that is used for contacting the superabsorbent with the surface crosslinking solution, and preferably the solution of the anti-microbial agent is added concurrently with the surface-crosslinking solution and any other additive that is added at this step, such as for example the polyvalent metal salt described above. In this context, “concurrently” means “in the very same piece of equipment”, but does not necessarily mean “through the very same nozzle or set of nozzles”. Separate nozzles or sets of nozzles are generally used to avoid interferences or reactions between the anti-microbial agent and the surface-crosslinking agent, but in cases where such interferences can be excluded, spraying one solution that comprises the anti-microbial agent, the surface-crosslinking agent and a polyol (that even may be part of or even constitute the crosslinking agent if the curing conditions lead to the formation of surface crosslinks by the particular polyol) through one nozzle or one set of nozzles is possible and if so, preferred for simplicity of apparatus and operation.

It is, however, also possible to add the solution of the anti-microbial agent separately from the surface crosslinker in a separate piece of equipment designed to contact solids with liquids such as those listed above for contacting the base polymer with the surface-crosslinking agent solution. The type of apparatus used for contacting the base polymer with the surface-crosslinking agent solution and the one used for contacting the base polymer with the anti-microbial agent solution may be different. If the antimicrobial agent is added separately from the surface crosslinking agent, it is added after the surface crosslinking agent, but prior to curing, preferably directly prior to curing.

The solvent used for the anti-microbial agent comprises a polyol.

The polyol is a compound having two or more hydroxy groups or a compound that releases a polyol under the conditions applied in the course of surface crosslinking. Typical polyols suitable as solvent component are linear or branched alkane diols such as ethylene glycol, 1,2-propanediol (propylene glycol), 1,3-propanediol, 1,2-butanediol, 1,3-butanediol, 1,4-butanediol, sec.-butylglycol, 1,2-pentanediol, 1,3-pentanediol, 1,4-pentanediol, 1,5-pentanediol or neopentylglycol. Other examples of suitable polyols are glycerol, trimethylolpropane, trimethylolethane or pentaerythritol. Compounds that release a polyol under the conditions applied in the course of surface crosslinking typically are polyols in which the hydroxi groups are masked or protected by masking or protecting groups. Examples of such masking or protecting groups are ethers or carbonates, and particular examples of compounds that are able to release a polyol under the conditions applied in the course of surface crosslinking are ethylene glycol dialkyl ethers, propylene glycol dialkyl ethers (such as the dimethyl or diethyl ethers thereof) 2-methoxidiethyl ether, ethylene carbonate or propylene carbonate. Mixtures of polyols are also suitable. Alkane diols are preferred, and 1,2-propanediol is most preferred.

Besides the polyol, the solvent may comprise other solvents or components such as for example water. Preferably, the solvent for the anti-microbial is a polyol or polyol mixture without further solvents or components.

Typically, the solution comprising the anti-microbial agent and the polyol comprises at least 20 wt.-%, preferably at least 40 wt.-% and more preferably at least 50 wt.-%, and typically not more than 90 wt.-%, preferably not more than 85 wt.-% and more preferably not more than 80 wt.-% of the anti-microbial agent. It further comprises generally at least 10 wt.-%, preferably at least 15 wt.-% and more preferably at least 20 wt.-% and generally not more than 80 wt-%, preferably not more than 60 wt.-% and more preferably not more than 50 wt.-% of polyol. The percent amounts of the components of the solution always add up to 100%.

A very suitable anti-microbial agent solution to be added concurrently with or directly after coating the superabsorbent with surface crosslinker solution , particularly in the case where the anti-microbial agent is triclosan, consists of 65 wt.-% of antimicrobial agent and 35 wt.-% of 1,2-propanediol.

After adding the surface-crosslinking agent solution, the antimicrobial agent solution and any other components to be added in the surface crosslinking step, surface crosslinking is completed by the thermal treatment described above.

After any drying step, it is advantageous but not absolutely necessary to cool the product after drying. Cooling can be carried out continuously or discontinuously, conveniently by conveying the product continuously into a cooler downstream of the dryer. Any apparatus known for removing heat from pulverulent solids can be used, in particular any apparatus mentioned above as a drying apparatus, provided it is supplied not with a heating medium but with a cooling medium such as for example with cooling water, so that heat is not introduced into the superabsorbent via the walls and, depending on the design, also via the stirrer elements or other heat-exchanging surfaces, but removed from the superabsorbent. Preference is given to the use of coolers in which the product is agitated, i.e., cooled mixers, for example shovel coolers, disk coolers or paddle coolers, for example Nara® or Bepex® coolers. The superabsorbent can also be cooled in a fluidized bed by blowing a cooled gas such as cold air into it. The cooling conditions are set such that a superabsorbent having the temperature desired for further processing is obtained. Typically, the average residence time in the cooler will be in general at least 1 minute, preferably at least 3 minutes and more preferably at least 5 minutes and also in general not more than 6 hours, preferably 2 hours and more preferably not more than 1 hour, and cooling performance will be determined such that the product obtained has a temperature of generally at least 0° C., preferably at least 10° C. and more preferably at least 20° C. and also generally not more than 100° C., preferably not more than 80° C. and more preferably not more than 60° C.

Superabsorbents are optionally also treated with a cohesion control agent to facilitate handling. A cohesion control agent is a non-aqueous liquid having a viscosity of at least 20 mPas, preferably at least 30 mPas, more preferably at least 40 mPas and most preferably at least 80 mPas, and generally not more than 1 000 mPas, preferably not more than 700 mPas, all at 20° C. A suitable cohesion control agent is at least one agent of this viscosity selected from the group formed by alcohols, poly glycols, silicon oils, hydrophilic modified silicon oils and paraffin oils.

These cohesion control agents and methods of adding them to superabsorbent are known per se. Generally, methods and apparatus suitable for a step of adding cohesion control agents are those described above for applying surface crosslinking agents, and in particular the apparatus described above as cooling equipment. Examples of suitable alcohols are 1,2-propylene glycol, 1,3-propane diol, 1,2-, 1,3- and 1,4-butandiol or glycerine. Examples of suitable polyglycols are poly ethylene glycols, poly propylene glycols or poly butylene glykols. Generally, these have a molecular mass of not more than 5 000 g/mol, preferably not more than 3 000 g/mol and more preferably not more than 2000 g/mol.

Preferred cohesion control agents are 1,2-propylene glycol, poly ethylene glycols with an average molecular weight of less than 1 500 g/mol, silicon oil, and hydrophilic modified silicon oil.

It is possible to apply more than one type of cohesion control agent. In general, the total amount of cohesion control agent added to a particular superabsorbent is adjusted to achieve the desired product properties, and to obtain a product that flows freely from a transport container such as a “big bag” into a feeding device. The optimum quantity of cohesion control agent depends on the type of superabsorbent and in particular on the type and amount of permeability enhancing agent. Typically, cohesion control agents are used in an amount of at least 100 wt.-ppm, preferably at least 200 wt.-ppm, more preferably at least 300 wt.-ppm and generally at most 5 000 wt.-ppm, preferably at most 3 000 wt.-ppm and more preferably at most 1 500 wt.-ppm, in each case based on the total weight of material.

The cohesion control agent is preferably applied to the polymer after surface crosslinking, and after the addition of permeability enhancing agent. Most preferably, the cohesion control agent is added after the heat treatment step applied during surface crosslinking or after a heat treatment step applied in the course of addition of permeability enhancing agent. It may be convenient to apply the cohesion control agent during a cooling step following surface crosslinking and addition of permeability enhancing agent, depending on whether the cooler provides sufficient mixing quality. It is always possible to add the cohesion control agent in a separate step, usually in a mixer, and preferably after surface crosslinking and adding a permeability enhancing agent. In some cases, it is possible to apply the cohesion control agent during surface crosslinking or addition of permeability enhancing agent, contingent upon inertness of the cohesion control agent during surface crosslinking.

Adding cohesion control agent usually necessitates no subsequent heating step. If a heating step should be necessary due to some special circumstances, care has to be taken to avoid any temperatures high enough for reaction between the cohesion control agent and the polymer.

A second method to add antimicrobial agent to the superabsorbent is contacting the superabsorbent with a solution comprising the anti-microbial agent and a polyalkylene glycol of a molecular mass between 200 and 5 000 g/mol after completion of surface crosslinking. To this end, a solution of the anti-microbial agent in a solvent that comprises the polyalkylene glycol is added to the superabsorbent. The solution can be added in any known way to achieve a coating of a solution on particles. Any method known or described above to achieve this with respect to a solution of the surface crosslinker or a cohesion control agent is feasible for the solution of the anti-microbial agent as well. Spraying the solution onto the superabsorbent is preferred. Preferably, this solution of the anti-microbial agent is added in the same type of apparatus that is used for contacting the superabsorbent with a cohesion control agent, and in the case where a cohesion control agent is added to the superabsorbent, preferably the solution of the anti-microbial agent is added concurrently with the cohesion control agent and any other additive that is added at this step. In this context, “concurrently” means “in the very same piece of equipment”, but does not necessarily mean “through the very same nozzle or set of nozzles”. Separate nozzles or sets of nozzles are generally used to avoid interferences or reactions between the anti-microbial agent and the cohesion control agent, but in cases where such interferences can be excluded, spraying one solution that comprises the anti-microbial agent and the cohesion control agent and a polyalkylene gylcol (that even may be part of the cohesion control agent or in fact constitute the cohesion control agent) through one nozzle or one set of nozzles is possible and if so, preferred for simplicity of apparatus and operation.

It is, however, also possible to add the solution of the anti-microbial agent separately from the cohesion control agent in a separate or, where no cohesion control agent is added, a dedicated piece of equipment designed to contact solids with liquids such as those listed above for contacting the base polymer with the surface-crosslinking agent solution or a cohesion control agent. The type of apparatus used for contacting the superabsorbent with the cohesion control agent and the one used for contacting the superabsorbent with the anti-microbial agent solution may be different. If the antimicrobial agent is added separately from the cohesion control agent, it is added prior to or after the cohesion control agent.

The solvent used for the anti-microbial agent in this second method comprises a polyalkylene glycol. The most common polyalkylene glycols are compounds that are technically produced by ring-opening polymerisation of alkylene oxides in the presence of water or an alcohol to yield compounds of the general formula

HO—(CHR¹—CHR²—O)ₙ—H

where R1 and R2 are the substituents of the alkylene oxide(s) used. Typically, R¹ and R² are (independently for each recurring unit) selected from H or an alkyl group, in particular methyl, ethyl, propyl. Preferably, one of R¹ and R² is H and the other H or methyl, ethyl or propyl. It is quite common that the polyalkylene glycol is derived from one type of alkylene oxide only, for example polyethylene glycol (R¹=R²=H), poly-propylene glycol or polybutylene glycol. Polyethylene glycol is preferred.

As a rule of thumb, the index n is generally at least 5 and preferably at least 6. Further, it is not more than 110, preferably not more than 85 and more preferably not more than 70. It is far more usual and precise, however, to refer to these polyalkylene glycols in terms of their average molecular weight since in the course of their production, there will always result a mixture of polyalkylene glycols with different n values. Consequently, a given amount of polyalkylene oxide always is a mixture of molecules of different chain lengths. The polyalkylene glycols to be used in the process of the present invention generally have a molecular weight of at least 200 g/mole, preferably 250 g/mole and more preferably at least 300 g/mole and generally not more than 5 000 g/mole, preferably not more than 3000 g/mole and more preferably not more than 1500 g/mole. These molecular weights naturally are average molecular weights.

It is also possible to use branched polyalkylene glycols that are produced by reacting polyols with alkylene oxides. Examples are the products of the ring-opening polymerisation of alkylene oxides, in particular of ethylene oxide, propylene oxide or a mixture thereof in the presence of glycerol or trimethylolpropane. For those compounds, the number of alkylene oxide units added to the core polyol in total (i.e., the sum of all alkylene oxide units in any of the polyalkylene oxide chains attached to the former hydroxy groups of the core polyol) is at least 3, preferably at least 5 and more preferably at least 6. Further, it is not more than 110, preferably not more than 85 and more preferably not more than 70.

A suitable polyalkylene glycol is polyethylene glycol of an average molecular weight of about 400 g/mole that is generally known and traded as “PEG-400”.

Besides the polyalkylene glycol, the solvent may comprise other solvents or components such as for example water. Preferably, the solvent for the anti-microbial is a polyalkylene glycol or polyalkylene glycol mixture without further solvents or components.

Typically, the solution comprising the anti-microbial agent and the polyalkylene glycol comprises at least 20 wt.-%, preferably at least 30 wt.-% and more preferably at least 40 wt.-%, and typically not more than 80 wt.-%, preferably not more than 70 wt.-% and more preferably not more than 60 wt.-% of the anti-microbial agent. It further comprises generally at least 20 wt.-%, preferably at least 30 wt.-% and more preferably at least 40 wt.-% and generally not more than 80 wt-%, preferably not more than 70 wt.-% and more preferably not more than 60 wt.-% of polyalkylene glycol. The percent amounts of the components of the solution always add up to 100%.

A very suitable anti-microbial agent solution to be added after surface crosslinking, particularly in the case where the anti-microbial agent is triclosan, consists of 50 wt.-% of antimicrobial agent and 50 wt.-% of PEG-400.

The anti-microbial agent may partly be added in the course of surface crosslinking and partly after surface crosslinking to achieve the desired end content of surface crosslinking agent. It is preferred to add the anti-microbial agent as part of the surface crosslinking solution, using the polyol not only as solvent for the anti-microbial agent, but also as surface crosslinking agent, and/or as part of the cohesion control agent, using the polyalkylene glycol not only as solvent for the anti-microbial agent, but also as cohesion control agent. In this way, a rather high amount of anti-microbial agent can be applied without necessity to use further auxiliaries, solvents or process steps.

Optionally, the superabsorbent is provided with further customary additives and auxiliary materials to influence storage or handling properties. Examples thereof are colorations, opaque additions to improve the visibility of swollen gel, which is desirable in some applications, surfactants or the like. Similarly, a final water content can be set for the superabsorbent, if desired, by adding water. These additives and auxiliary materials can each be added in separate processing steps, but one convenient method may be to add them to the superabsorbent in the cooler, for example by spraying the superabsorbent with a solution or adding them in finely divided solid or in liquid form, if this cooler provides sufficient mixing quality.

The surface-crosslinked superabsorbent produced using the process of the instant inventions is optionally ground and/or sieved in a conventional manner. Grinding is typically not necessary, but the sieving out of agglomerates which are formed or undersize is usually advisable to set the desired particle size distribution for the product. Agglomerates and undersize are either discarded or preferably returned into the process in a conventional manner and at a suitable point; agglomerates after comminution. The superabsorbent particle size is preferably not more than 1000 μm, more preferably not more than 900 μm, most preferably not more than 850 μm, and preferably at least 80 μm, more preferably at least 90 μm and most preferably at least 100 μm. Typical sieve cuts are for example 106 to 850 μm or 150 to 850 μm.

The process of the present invention is also suitable for producing superabsorbents that are not in powder form. In that case, the anti-microbial agent still is added to the web comprising superabsorbent particles or to the superabsorbent fibres or whatever other form the particular superabsorbent takes, as described above in the course of surface crosslinking and/or afterwards.

We have further found superabsorbent produced by the process of the present invention and hygiene articles comprising the superabsorbent produced by the process of the present invention. Hygiene articles in accordance with the present invention are for example those intended for use in mild or severe incontinence, such as for example inserts for severe or mild incontinence, incontinence briefs, also diapers, training pants for babies and infants or else feminine hygiene articles such as liners, sanitary napkins or tampons. Hygiene articles of this kind are known. The hygiene articles of the present invention differ from known hygiene articles in that they comprise the superabsorbent of the present invention. We have also found a process for producing hygiene articles, this process comprising utilizing at least one superabsorbent of the present invention in the manufacture of the hygiene article in question. Processes for producing hygiene articles using superabsorbent are otherwise known.

The present invention further provides for the use of the composition of the present invention in training pants for children, shoe inserts and other hygiene articles to absorb bodily fluids. The composition of the present invention can also be used in other technical and industrial fields where liquids, in particular water or aqueous solutions, are absorbed. These fields are for example storage, packaging, transportation (as constituents of packaging material for water- or moisture-sensitive articles, for example for flower transportation, also as protection against mechanical impacts); animal hygiene (in cat litter); food packaging (transportation of fish, fresh meat; absorption of water, blood in fresh fish or meat packs); medicine (wound plasters, water-absorbing material for burn dressings or for other weeping wounds), cosmetics (carrier material for pharmachemicals and medicaments, rheumatic plasters, ultrasonic gel, cooling gel, cosmetic thickeners, sun protection); thickeners for oil-in-water and water-in-oil emulsions; textiles (moisture regulation in textiles, shoe inserts, for evaporative cooling, for example in protective clothing, gloves, headbands); chemical engineering applications (as a catalyst for organic reactions, to immobilize large functional molecules such as enzymes, as adhesion agent in relation to agglomerations, heat storage media, filter aids, hydrophilic component in polymeric laminates, dispersants, superplasticizers); as auxiliaries in powder injection moulding, in building construction and engineering (installation, in loam-based renders, as a vibration-inhibiting medium, auxiliaries in tunnel excavations in water-rich ground, cable sheathing); water treatment, waste treatment, water removal (deicing agents, reusable sandbags); cleaning; agritech (irrigation, retention of melt water and dew deposits, composting additive, protection of forests against fungal/insect infestation, delayed release of active components to plants); for firefighting or for fire protection; coextrusion agents in thermoplastic polymers (for example to hydrophilize multilayered films); production of films and thermoplastic mouldings able to absorb water (for example rain and dew water storage films for agriculture;

superabsorbent-containing films for keeping fruit and vegetables fresh which are packed in moist films; superabsorbent-polystyrene coextrudates, for example for food packaging such as meat, fish, poultry, fruit and vegetables); or as carrier substance in formulations of active components (pharma, crop protection).

### Superabsorbent Property Test Methods

### Centrifuge Retention Capacity (CRC)

The method for determination of the Centrifuge Retention Capacity (CRC) is described in US patent application no. US 2002/0 165 288 A1, paragraphs [0105] and [0106].

Moisture content

The moisture content is determined according to EDANA (European Disposables and Nonwovens Association, Avenue Eugene Plasky, 157, 1030 Brussels, Belgium, www.edana.org) recommended test method No. 430.2-02 “Moisture content”, available from EDANA.

Particle size distribution

Particle size distribution is determined according to EDANA recommended test method No. 420.2-02 “Particle size distribution”, available from EDANA.

1. A process for producing superabsorbents having a coating of an antimicrobial agent comprising contacting the superabsorbent with a solution comprising the antimicrobial agent and a polyol concurrently with or immediately after contacting the superabsorbent with the a surface-crosslinking agent and prior to a curing step that completes surface crosslinking, and/or contacting the superabsorbent with a solution comprising the antimicrobial agent and a polyalkylene glycol of a molecular mass between 200 and 5 000 g/mol after completion of surface crosslinking.

2. The process of claim 1 wherein the antimicrobial agent is triclosan.

3. The process of claim 1 wherein the polyol is a glycol.

4. The process of claim 3 wherein the polyol is 1,2-propanediol.

5. The process of claim 1 wherein the polyalkylene glycol is polyethylene glycol or polypropylene glycol.

6. The process of claim 5 wherein the polyalkylene glycol is polyethylene glycol of an average molecular weight of 400 g/mole.

7. A superabsorbent having a coating of an antimicrobial agent that is produced by the process of claim 1.

8. A hygiene article comprising the superabsorbent of claim 7.

9. The process of claim 2 wherein the polyol is a glycol.

10. The process of claim 9 wherein the polyol is 1,2-propanediol.

11. The process of claim 2 wherein the polyalkylene glycol is polyethylene glycol or polypropylene glycol.

12. The process of claim 11 wherein the polyalkylene glycol is polyethylene glycol of an average molecular weight of 400 g/mole.
